(19) United States
(12) Patent Application Publication
(10) Pub. No.: US 2021/0252149 A1
(43) Pub. Date: Aug. 19, 2021

(54) Composition and Methods for Treating Infectious Agents Using Pathogen-specific Antibodies

(71) Applicant: Anubis Bio Corporation, Oviedo, FL (US)

(72) Inventor: Thomas A. Schultz, Oviedo, FL (US)

(21) Appl. No.: 17/180,351

(22) Filed: Feb. 19, 2021

Related U.S. Application Data
(60) Provisional application No. 62/978,598, filed on Feb. 19, 2020.

(51) Int. Cl. A61K 39/42; A61P 31/14; A61P 31/20; A61P 31/04; A61K 9/00; A61K 39/40; A61K 35/20

(52) U.S. Cl. CPC A61K 39/42; A61P 31/14; A61P 31/20; A61K 35/20; A61K 9/006; A61K 39/40; A61P 31/04

(57) ABSTRACT

A composition for treatment of mucosal infection in an animal by application to a mucous membrane includes a mixture of IgY antibodies specific for a plurality of antigens obtained from a plurality of organisms, with the plurality of organisms able to cause infection of the mucous membrane. A protective matrix including non-hyperimmune colostrum can be combined with the mixture of IgY antibodies, with the protective matrix forming at least 20% by weight of the composition.

This application claims the benefit of co-pending U.S. Provisional Patent Application Ser. No. 62/978,598 filed on Feb. 19, 2020 which is hereby incorporated by reference in its entirety.

### FIELD OF THE INVENTION

Compositions and methods for treatment of tissues, membranes, or biofilms, including mammalian mucosal membranes are described. In some embodiments, compositions include an effective combination of avian-sourced antibodies.

### BACKGROUND

Antibodies, both naturally occurring and their synthetic analogues, are known therapeutic agents in animals. Antibodies bind a portion of the antigen (the antigenic determinant or epitope) with the antigen combining site on the antibody. Antibodies are capable of high degrees of specificity enabling targeted application to specific pathogens. However, this high specificity can lead to excessively limited binding attributes, where agents or antigens that are functionally identical (for example, cause the same disease or symptoms) do not react identically with the immunoreagent or immunotherapeutic. Cross-reactivity on the other hand is the reaction between an antigen and an antibody that was generated against a similar but different antigen. Controlled cross-reactivity may constructively be used to broaden the binding range of the antibody, enabling broad spectrum protection against pathogens other than those specifically targeted.

In mammalian species, immunity to pathogens is transferred from mother to offspring via maternal antibodies provided by the placenta or colostrum. The mother is able to transfer only those antibodies that were built up by her body due to natural exposure or vaccinations. However, her level of transfer of antibodies is influenced by how recently exposure to specific pathogens occurred. If the maternal colostrum contains an insufficient quantity of antibodies specific for certain pathogens, the neonate will have a deficient level of immunity for those diseases.

Colostrum has evolved naturally in mammals specifically to deliver its components to neonates into and through the gastrointestinal tract in a very concentrated low-volume form. Colostrum is known to contain antibodies including IgA, IgG, and IgM. Other components of colostrum include lactoferrin, lysozyme, lactoperoxidase, complement, and proline-rich polypeptides (PRP). Other components in colostrum have been shown to protect and support the antibody activity in the gastrointestinal tract.

The antibodies and cofactors in colostrum can provide a passive immunity to the recipient. Normally, antibodies and cofactors are passed to the neonate from the mother and provide the first protection against pathogens. Growth factors in colostrum also stimulate the development and repair of the gut. Other components in colostrum help protect maternal antibodies on their journey through the digestive system and support antibody activity in the intestine. This is a particularly important function in bovine species which have a more extensive gastrointestinal tract than many other mammals.

Colostrum is naturally designed to serve as a protective/reactive matrix within a gastrointestinal environment. It helps to regulate the intestinal environment, rendering it hostile to foreign pathogens. As an example, colostrum contains lactoferrin, an iron-binding protein that prevents bacteria and viruses from obtaining iron necessary for replication. Colostrum also selectively fertilizes certain probiotic species that, in turn, help to ward off infection. Colostrum is a source of two major growth factors, Transforming Growth Factors (TGF) alpha and beta, as well as a source of Insulin-Growth Factors 1 and 2. These factors promote tissue repair and development. Colostrum is also a source of Hepatocyte Growth Factor (HGF, also known as “scatter factor”), which stimulates the growth and expansion of intestinal wall cells.

Specific antibody production via immunization of an avian species, for example, chickens, is well documented. When immunized with an appropriate antigen, the hen responds by producing IgY antibodies which are concentrated in the egg yolk for use by the chick during the first weeks of life. Transfer of IgY antibodies from the yolk to the developing chick substitutes for the mammal delivering antibodies to the neonate in the form of colostrum.

IgY antibodies from avian eggs have been shown to be effective against pathogens residing in the gastrointestinal tract of mammals. Unfortunately, orally delivered antibody therapeutic effectiveness is diminished by passage through the stomach and exposure to gastric acid and digestion enzymes. Improved delivery mechanisms for IgY antibodies from avian eggs are needed.

### SUMMARY

A composition for treatment of mucosal infection in an animal by application to a mucous membrane includes a mixture of IgY antibodies specific for a plurality of antigens obtained from a plurality of organisms, with the plurality of organisms able to cause infection of the mucous membrane. A protective matrix including non-hyperimmune colostrum can be combined with the mixture of IgY antibodies, with the protective matrix forming at least 20% by weight of the composition.

Another embodiment is a method of treating or preventing infection by providing an antibody mixture including a non-hyperimmune colostrum and antibodies produced by an avian animal to act against an organism that infects a mucous membrane. Treatment involves applying the antibody mixture to the mucous membrane. In some embodiments, the composition can be administered as a solid, a powder, a powder suspended liquid, a cream, as a liquid, by mist, aerosol, or spray, or coated on an object insertable into an animal.

Another embodiment is a composition for therapeutic treatment of biofilms. The composition can include a mixture of IgY antibodies specific for a plurality of antigens obtained from a plurality of biofilm forming organisms, with the plurality of biofilm forming organisms being able to cause infection. A protective matrix including non-hyperimmune colostrum can be combined with the mixture of IgY antibodies, with the protective matrix including at least 20% by weight of the composition.

Another embodiment is a method of treating or preventing biofilm formation by providing an antibody mixture including a non-hyperimmune colostrum and antibodies produced by avian animal to act against a biofilm forming organism. Treatment involves applying the antibody mixture to the biofilm or to an object, membrane, or tissue that might support a biofilm.

### BRIEF DESCRIPTION OF THE DRAWINGS

In order that the advantages of the invention will be readily understood, a more particular description of the invention briefly described above will be rendered by reference to specific embodiments illustrated in the appended drawing. Understanding that this drawing depicts only a typical embodiment of the invention and is not therefore to be considered limiting of its scope, the invention will be described and explained with additional specificity and detail through use of the accompanying drawing.

FIG. 1 is a schematic drawing of a method of producing the disclosed composition;

FIG. 2 is a flow chart describing a method of producing the disclosed composition; and

FIG. 3 is a flow chart describing a method of treating a subject using the disclosed composition.

### DETAILED DESCRIPTION OF THE INVENTION

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “organism” means a form which may have a single cell or multiple cells, and which include bacteria, viruses, and parasites.

As used herein, “disease” means an illness caused, at least in part, by a pathogen breaching the mucous membrane of a subject.

As used herein, “subject” means a human or animal, including mammals.

As used herein, “pathogen” means an organism which causes disease or untoward effects in a host and which include bacteria, viruses, and parasites.

While this invention is susceptible of embodiment in many different forms, there is shown in the drawings, which will herein be described in detail, specific embodiments with the understanding that the present disclosure is to be considered as an exemplification of the principals of the invention and is not intended to limit the invention to the illustrated embodiments.

In one embodiment, specifically targeted avian-sourced antibodies can be used to introduce effective passive immunity to a mammalian subject in need thereof. The antibodies may be created to react with specific pathogenic and/or toxin targets associated with diseases caused by infection of mucous membranes.

The use of the described compositions and methods can enable one or more of (a) customized design of antibodies for specified or targeted diseases; (b) dose controlled formulation of a variety of mixtures of components, which may be tuned or adjusted for effect; (c) dose controlled formulation that provides specified components in excess of normal physiological levels that may be achieved in natural systems; (d) use complex component interactions to create a systems effect that emulates a native immune system response; (e) creation of a preconditioned or potentiated immune response that can be administered in its potentiated state, and subsequently activated by the presence of the target pathogens, toxins, disease state, or syndrome; (f) creation of formulations that have a defined specificity or broad-spectrum effect, to match the needs of the specific target disease state or syndrome; and (g) the creation of formulations that can be targeted for prophylaxis as well as for treatment.

In some embodiments, compositions and methods such as disclosed herein can be used for treatment of tissues, membranes, or biofilms, including mammalian mucosal membranes. Additionally, therapeutic compositions can be used in the prevention or treatment of a pathogen-induced disease in a subject wherein the pathogen causes the disease by breaching a mucous membrane in the subject. Methods of production of the therapeutic composition and methods of treating a subject to prevent or treat a pathogen-induced disease, wherein the disease is caused, at least in part, by breaching of a mucous membrane by a pathogen are also disclosed.

The mucosal membranes, also called the mucosa, are membranes that line many body cavities and tubular organs. Such membranes act as a barrier preventing foreign substances, including pathogens and particulate matter, from entering the body. Some mucous membranes secrete mucous which acts to prevent tissue dehydration as well as to trap pathogens and particulate matter which might otherwise gain entrance to deeper tissues.

Examples of locations of mucous membranes include the respiratory tract (e.g. the bronchial mucosa and nasal mucosa), the urogenital tract (e.g. the endometrium, the vaginal mucosa, and the penile mucosa), the gastrointestinal mucosa (e.g. the tongue, the frenulum of the tongue, the esophageal mucosa, the gastric mucosa, the intestinal mucosa, the oral mucosa, and the anal canal), the middle ear mucosa, and the palpebral conjunctiva of the eye.

Pathogens which infect the mucous membranes include viruses, bacteria, yeasts, and fungi. For example, the mouth, esophagus, and vagina are relatively frequently infected by fungi. While disease can occur due to direct infection of the mucous membrane, systemic disease can occur if the pathogen contacts or is able to breach the mucous membrane.

An example of disease caused by bacterial breach of the intestinal mucous membrane is typhoid fever. Salmonella enterica serotypes enter the gastrointestinal tract, typically through fecal contamination of food and water. These bacterial cells invade intestinal epithelium causing systemic disease. Tissues including the liver, spleen, gallbladder, and bone marrow may be infected once the bacteria cross the intestinal epithelium. Other strains of Salmonella, called non-typhoidal Salmonella, typically cause gastroenteritis. However, up to 5% of non-typhoidal Salmonella infections cause invasive, extra-intestinal systemic infections.

Advantageously, biofilms, membranes, tissues, or mucous membranes outside of the gastrointestinal tract are not ordinarily subjected to acid or digestive enzymes that can destroy or inactivate the disclosed therapeutic agents. Consequently, these mucous membranes represent sites for antibody therapeutics which may complement or replace small molecule pharmaceutical products.

In other embodiments, unwanted biofilms can be treated with the disclosed therapeutic agents to reduce or destroy infectious agents. A biofilm comprises any syntrophic consortium of microorganisms in which cells stick to each other and are supported by a living or non-living surface. These attached cells can become embedded within a thin film or film having three dimensional structure that is formed with a slimy, hydrogel, or mucus-like extracellular matrix composed of extracellular polymeric substances including polysaccharides, proteins, lipids, or DNA. Biofilms may form on supports such as living animal membranes, mucosal membranes, tissues, or bones or teeth (e.g. dental plaque), and often provide a long term reservoir for infectious agents. Additionally, biofilms can form and be supported on non-living objects and surfaces that are insertable into an animal. Reducing or eliminating such biofilms can reduce or eliminate risk of infections in an animal with mucosal infections, injuries to mucosal membranes, or to those animals suffering from reduced immunological functionality.

In some embodiments, even non-living, inorganic, plastic, or man-made materials that might support unwanted biofilms, including biofilms containing viruses, bacteria, yeasts, fungi, or animal cells, can be treated with the disclosed therapeutic agents to reduce or destroy infectious agents. In some embodiments, this therapeutic application can be made to insertable objects such as tubing, catheters, or implanted sensors or monitors to reduce or eliminate biofilms. For example, therapeutic applications can be made to a vein inserted device that could support unwanted biofilms, reducing the risk of infectious agent biofilms and providing therapeutic agents directly to membranes or tissues directly damaged by vein insertion. Alternatively, tubing for respiratory support, gastrointestinal sampling, or internal body inspection can be coated or include therapeutic agents that similarly reduce or eliminate unwanted biofilms and providing therapeutic agents directly to membranes (including mucosal membranes) or tissues directly or indirectly damaged by tubing insertion.

One embodiment is a method to create a targeted antibody-based formulation, where the antibodies use a controlled form of cross-reactivity to multiple clusters of related target antigens. The utility of such antibody formulations may include providing broad-spectrum therapeutic interventions under conditions where the class of causative agent, but not the precise or specific causative agent is known or suspected or under circumstances where multiple (mixed) causative agents are active.

One embodiment takes advantage of both the specificity and cross-reactive attributes of antibodies to generate a multi-component in situ immune response. In this embodiment, antibodies are designed to bind to several closely related epitopes that represent a structurally related cluster of antigens. These antigens may differ markedly in other respects, and may originate from diverse sources, organisms, or species.

One embodiment involves the method of using immune factors, (for example antibodies), where the immune factors have specificity to a class of related antigens and are cross-reactive to different instances of members of that class. There exists a degree of structural similarity in related clusters of target antigens, without regard to the organism or pathogen that is the source of the antigen. Similarity in structure can result in a phenomenon known as “cross-reactivity” (the steric binding of a reactive molecule to an antigen other than the antigen intended). Cross-reactivity is often unintentional and is considered a source of error and non-specificity. However, in this embodiment the extent and degree of cross-reactivity is controlled by various means to limit and channel its expression so as to provide desired characteristics.

This treatment confers passive immunity to subjects. The nature of the treatment makes the associated risk factors comparable to that of eating food from the source where the antibodies were harvested (e.g., risk factors would be similar to that of eating an egg). This is an effective treatment with less toxicity than the currently available alternative medicines.

One embodiment relates to use in the production of a broad spectrum therapeutic. One method for producing this type of reactive formulation involves the production of polyclonal antibodies harvested from an appropriately immunized animal. They are typically harvested from the serum, colostrum, or eggs of an immunized animal.

In this embodiment, the polyclonal antibody mixture is a mixture of immunoglobulin molecules which interact with a specific antigen, or group of antigens, recognizing a range of different epitopes. It is possible to have multiple antibodies for a single antigen (binding to different active sites) or for a single antibody to bind to multiple antigens. This contrasts with monoclonal antibodies, which are identical and monospecific; being produced by one type of immune cell that are all clones of a single parent cell.

The antibodies used in this therapeutic may be collected from serum, plasma, colostrum, milk, eggs, or other suitable biologically derived fluid, or from cell culture media, supernatant.

The antibodies used in this therapeutic may be treated in any suitable manner to prepare for formulation and use, including but not limited to separations, plasmapheresis, spray dry or other drying processes, lyophilization, pasteurization, and preservation methods. The antibodies used in this therapeutic may be treated, concentrated, separated, or purified in various ways depending upon their final intended use.

There is a clear need for low cost and effective treatments for many pathogens which gain access to a subject through a mucous membrane, and antibodies which are directly administered to a mucous membrane are candidates for this role. In addition to demonstrated efficacy, orally and topically administered antibodies are typically non-immunogenic so do not result in an adverse immunological response in the subject receiving the composition. They are typically well tolerated with no adverse side effects reported and comparatively no different reactions than a comparable ingested food product. Notably several products containing orally administered antibody have received GRAS (Generally Recognized as Safe) certification by the U.S. Food and Drug Administration.

The disclosed composition includes IgY antibodies may be derived from eggs laid by chickens or other avian species (egg-laying hens). These hens have been immunized against one or more pathogens which cause disease in humans, animals, or other organisms. In the example in which chickens are used to produce the eggs, the chickens may be any domestic bird of the subspecies Gallus domesticus. Examples include, but are not limited to, the following breeds of Gallus domesticus: Rhode Island Red, Leghorn, Australorp, Lohmann Brown Classic, Sussex, Golden Comet, Marans, Plymouth Rock, Barnevelder, Buff Orpington, Ameraucana, La Brese, and Hamburg. These examples are breeds of chickens which are known to be prolific egg producers. However, other breeds of chicken and other avian species are within the scope of this disclosure. In some embodiments, the chickens may comprise breeds meeting safety and process regulations for animal consumption as promulgated by relevant government authority (for example, the United States Department of Agriculture (“USDA”)).

Prior to egg collection, the hens may be immunized with a vaccine comprising at least one antigen which initiates production of antibodies directed against one or more pathogens which cause disease by breaching a mucous membrane. The vaccine may be produced by any method known in the art. Examples include attenuated live vaccines, modified live vaccines, chemically altered vaccines, killed vaccines, toxoid vaccines, DNA vaccines, mRNA vaccines, subunit vaccines, recombinant vaccines, polysaccharide vaccines, and conjugate vaccines. The vaccines may be directed against viruses, bacterial pathogens, parasites, yeasts, or molds. The vaccines may also be directed against adhesins or toxins produced by pathogens. In some embodiments, the vaccines may include one or more adjuvants which enhance the immunogenicity of the vaccine.

In some embodiments, hens may be immunized with vaccines which include a live, wild-type pathogen. Vaccines are typically created using pathogens which have been rendered less virulent (by modifying or killing it) and unlikely to result in clinical illness in the organism receiving the vaccine. However, a pathogen that causes disease in a human or animal may not cause any illness in an avian species although the bird may produce antibodies against it. Consequently, the hens remain healthy but are still able to raise antibodies against the vaccine components which bind to the pathogens within an infected subject. An advantage to using live, wild type pathogens in vaccines is that they are more immunogenic than their attenuated counterparts resulting in greater antibody production. Accordingly, adjuvants may not be required in these vaccines. As another advantage, use of the disclosed methods allows expansion of the universe of available antibodies that can be quickly and safely employed against biofilm, membrane, mucosal membrane, or tissue infections. Typically, egg-generated antibodies are non-pathogenic in mammals (i.e. Generally Recognized As Safe (GRAS)). This enables the use of a wide selection of antigens for therapeutic application including: highly virulent (to mammals) antigens, untested or novel antigens, unidentified antigens (e.g. collected from infected mammal tissue or fluids), or highly specific antigens (e.g. those collected from a specific patient).

Disease-causing pathogens against which the hens may be immunized include, but are not limited to, Streptococcus zooepidem, Bordetella bronchiseptica, Pasteurella, Haemophilus parasuis, Bovine Respiratory Syncytial Virus (BRSV), Parainfluenza 3 (PI3), Adenovirus, Bovine Viral Diarrhea Virus (BVDV), Infectious Bovine Rhinotracheitis (IBR)), Pasteurella multocida, Mannheimia haemolytica, Histophilus somni, Mycoplasma bovis, Parasitic organisms (e.g. lungworm), fungal organisms (e.g. Aspergillus), Pseudomonas aeruginosa, Staphylococcus hyicus, Escherichia coli, Trueperella pyogenes, Fusobacterium necrophorum, Streptococcus zooepidemicus, Klebsiella pneumoniae, Streptococcus equi subsp zooepidemicus, Candida albicans, Streptococcus equi, zooepidemicus, Yersinia enterocolitica, Salmonella Typhimurium, Pasteurella multocida, Streptococcus equi zooepidemicus, Actinobacillus equuli, Pasteurella spp, β-haemolytic streptococci, Trueperella pyogenes, Actinobacillus suis, Bacteroides spp., Clostridium spp., Ureaplasma ureolyticum, Mycoplasma spp, Taylorella equigenitalis, Pasteurella multocidia (PMt), Streptococcus suis, Mannheimia haemolytica, Pasteurella multocida, Histophilus somni, Mycoplasma bovis, Streptococcus spp., Corynebacterium renale, Corynebacterium cystitidis, Corynebacterium pilosum, Pseudomonas aeruginosaz, Arcanobacterium spp., Trueperella spp., Salmonella, Clostridium difficile, Clostridium perfringens, Lawsonia intracellularis, and Neorickettsia risticii.

Examples of diseases which are the result of pathogens associated with a mucous membrane and which may be treated or prevented using the disclose therapeutic include, but are not limited to, the following: oral thrush, canker sores, bronchitis, endometritis, candidiasis, pharyngitis, gastroenteritis, giardia infection, chlamydia, gonorrhea, rhinitis, sinusitis, vaginitis, vaginal yeast infection, otitis, otitis media, group B streptococcus infection of the vagina or rectum, oral or genital herpes, vaginal yeast infection, cystitis, urinary tract infection, proctitis, paracoccidioidomycosis, histoplasmosis, mucormycosis, candida, group A streptococcal infection of the pharynx, respiratory syncytial virus (RSV) infection, and anusitis.

Antibodies used in this therapeutic may be administered as a solid, a powder, a powder suspended liquid, a cream, as a liquid, by mist, aerosol, or spray, coated on an object insertable into an animal, or any method suitable to their immunogenic or biologically or immunologically reactive characteristics provided that the method introduces the therapeutic to an object, biofilm, membrane, mucous membrane, or tissue. Routes of administration may include, but are not limited to, oral, oropharyngeal, buccal, laryngeal, sublingual, endotracheal, transtracheal, nasal (e.g. drops, mists, aerosols, or sprays), intravaginal, ophthalmic (e.g. eye drops), or rectal (e.g. suppository or enema). In some embodiments, the therapeutic may be administered into the ear, for example, as ear drops. In some embodiments, the therapeutic may be administered through urethral methods, for example, through either internally or externally on a catheter which may be inserted into the urethra. In some embodiments, the therapeutic may be administered either internally or externally into the gastrointestinal tract through a nasogastric tube or by gastric gavage.

In some embodiments, each hen may be immunized with a single disease-causing pathogen. In this example, multiple hens may each be immunized with a different single disease-causing antigen and the eggs from each hen may be combined to result in a product which includes IgY antibodies against different antigens. In other embodiments, a single hen may be immunized with two or more disease-causing pathogens resulting in IgY antibodies against the different antibodies being present in the same egg.

An example of method of selecting groups of pathogens against which a hen may be vaccinated is based on known multiple causative organisms in diseases involving a mucous membrane. These causative organisms may be organized into common clusters of structurally related toxins or disease causing subunits or the organism, to which a series of broad-spectrum neutralizing antibodies may be created. When mixed into a formulation with clinically effective titers, these antibodies may be used as a broad-spectrum organism-independent therapeutic intervention for toxin-mediated disease.

By using this method of selecting pathogens for vaccination, it may not be necessary to know the precise pathogen which is causing disease in the subject to receive the composition. Also, when used prophylactically, the subject is protected against many disease-causing organisms. For example, the antibodies raised in the hens may use a controlled form of cross-reactivity to multiple clusters of related target antigens. There exists a degree of structural similarity in related clusters of target antigens, without regard to the organism or toxin that is the source of the antigen. By raising antibodies against antigens common to these related clusters, a broad-spectrum therapeutic intervention is created for use in situations where the class of causative agent, but not the precise or specific causative agent is known or suspected, or under circumstances where multiple (mixed) causative agents are active.

Furthermore, used of the described compositions and methods can provide an effective and immediate response to an emergent undifferentiated pathogen strain with a new combination of features, some of which already reside in other microbes. For example, new combinations of pathogen features result from random mutation, inclusion of DNA from other microbes, or antibiotic-mediated selective evolution. These events create new, highly virulent pathogen strains with limited response to existing medical treatments. These altered strains often result in high morbidity/mortality for months or years until a vaccine or other treatment is developed.

This approach takes advantage of both the specificity and cross-reactive attributes of the antibodies. In this embodiment, antibodies are designed to bind to several closely related epitopes that are present within a structurally related cluster of antigens. These antigens may differ markedly in other respects, and may originate from diverse sources, organisms, or species but have the common effect of causing disease process involving pathogen breach of a mucous membrane.

In this case the inoculant or immunogen is selected to a common or preserved component or region of the targeted antigen cluster, while ignoring the variable or distinguishing components or regions of the individual members of the cluster of related antigens. The method involves the preparation a vaccine which will be administered to the birds and which comprises an appropriate immunogen with characteristics that elicit the production of antibodies that are cross-reactive to desired instances of that epitope, but which are not reactive to other epitopes.

One example of this embodiment includes the production of antitoxin antibodies that are specifically reactive to clusters of structurally related toxins. These example antibodies would have effect without regard to the species originating the toxin. For example, the antibodies raised against the structurally related toxins may be neutralizing antibodies, capable of neutralizing or inactivating the biological activity of the target toxins.

Such a broad-spectrum neutralizing antibody could be used as disclosed herein to intervene in certain types of diseases where the toxin mediating the symptoms is one of a cluster of toxins without requiring knowledge of which organism was causative. Further, if a therapeutic according to the instant disclosure was prepared containing multiple antibodies in clinically effective amounts, the formulation could be used to intervene in cases where the active toxin responded to any of the antibodies in the mixture.

This method may be extended to include any number of toxin clusters, and to include broad-spectrum neutralizing antibodies against mediators of other toxin-like reactions (for example viral toxin-like phenomena), to create a broadly applicable intervention to disease. Using these antibodies to prepare a therapeutic as disclosed herein, symptoms and pathology may be managed or prevented without knowledge of the infectious causes, or in cases where there are multiple infectious causes. As previously noted, another advantage of the disclosed method relates to the wide range of available antibodies (including but not limited to those GRAS derived egge-generated antibodies) that can be quickly and safely employed against biofilm, membrane, mucosal membrane, or tissue infections. This enables the use of a wide selection of antigens for therapeutic application including: highly virulent (to mammals) antigens, untested or novel antigens, unidentified antigens (e.g. collected from infected mammal tissue or fluids), or highly specific antigens (e.g. those collected from a specific patient).

Protocols for immunizing the hens with the vaccine may be according to those known in the art for initiating antibody production in chickens. In an example, the hens may receive two or more vaccinations at least two weeks apart. In some embodiments, the vaccinations may begin when the hens are 18 weeks of age or older. Booster vaccines may be given to the hens 6 months after the first vaccination.

In some embodiments, the vaccines are administered to the hens subcutaneously. In other embodiments, the vaccines are administered through intramuscular, oral, intravenous, buccal, nasal, or dermal procedures.

After the immunization process, whole shell eggs may be collected from the hens. The yolks of these eggs contain concentrated IgY which bind to the one or more pathogens against which the laying chicken was vaccinated. In other embodiments, the yolk of the eggs may be isolated from the egg whites.

A dehydrated egg powder may be produced from the eggs (either whole shell or isolated yolks) according to procedures known in the art. In one embodiment, the eggs may be pan dried using commercial dehydrators suitable for liquid egg. In some embodiments the commercial dehydrators may meet food processing standards as promulgated by relevant regulatory bodies (for example, USDA). The drying temperature may be at least 138 F, but not to more than 150 F which is sufficient to pasteurize egg and dry to powder within 15 hours. The dehydrated egg product may then be ground produce a powder suitable for mixing.

In another embodiment, the eggs pay be spray dried. In this embodiment, the liquid eggs may be pasteurized at 140 F immediately prior to spray dry using dedicated food quality process equipment following relevant regulatory guidelines. The dried egg product may then be ground produce a powder suitable for mixing.

Additionally, other drying processes, lyophilization, pasteurization, and preservation methods may be used to process the eggs. Furthermore, the antibodies in the eggs may be concentrated, separated, or purified in various ways known in the art. The antibodies produced as disclosed herein may be purified, treated, or retained in the egg material for use in manufacturing the disclosed therapeutic.

In embodiments in which the therapeutic or prophylactic described herein is to be administered into the gastrointestinal tract, a protein carrier may be mixed with the antibody mixture. In some embodiment, the protein carrier may include non-hyperimmune colostrum, serum albumen, or other suitable protein which may protect the antibodies from the gastrointestinal environment.

One embodiment is a broad spectrum therapeutic or prophylactic antitoxin formulation comprising a mixture of broad-spectrum neutralizing antibodies, produced according to this method, for the purposes of allowing for effective administration across a wide range of unknown or undiagnosed conditions resulting in toxin mediated disease impacting the mucous membranes.

One embodiment is a broad spectrum therapeutic or prophylactic anti-pathogen formulation for administration to a subject, containing a mixture of broad-spectrum anti-pathogen antibodies produced according to this method.

One embodiment is a broad spectrum therapeutic or prophylactic anti-adhesin formulation for administration to a subject containing a mixture of broad-spectrum anti-adhesin antibodies produced according to this method.

One embodiment is a broad spectrum therapeutic or prophylactic formulation for administration to a subject containing a mixture of broad-spectrum antitoxin, anti-pathogen, and anti-adhesin antibodies produced according to this method.

One important limitation of using natural food-based products is that preparations are limited to the results allowed by natural processes. The disclosed combinations and methods allow for the selective addition of specific antibodies and general immune factors (formulations) that are significantly higher than physiological levels than can normally be achieved in nature. In some embodiments, various factors can be weighted in a manner that creates greater specificity to targeted diseases, pathogens, or toxins.

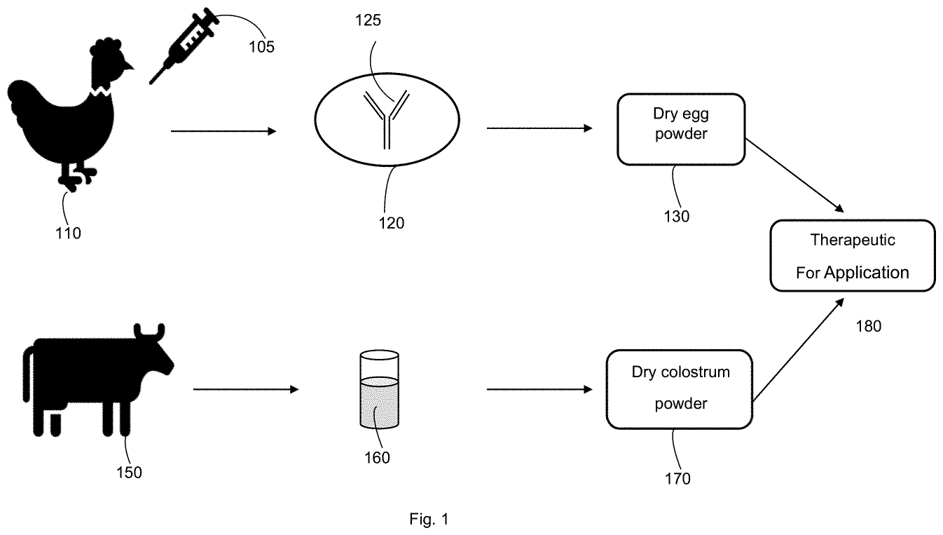

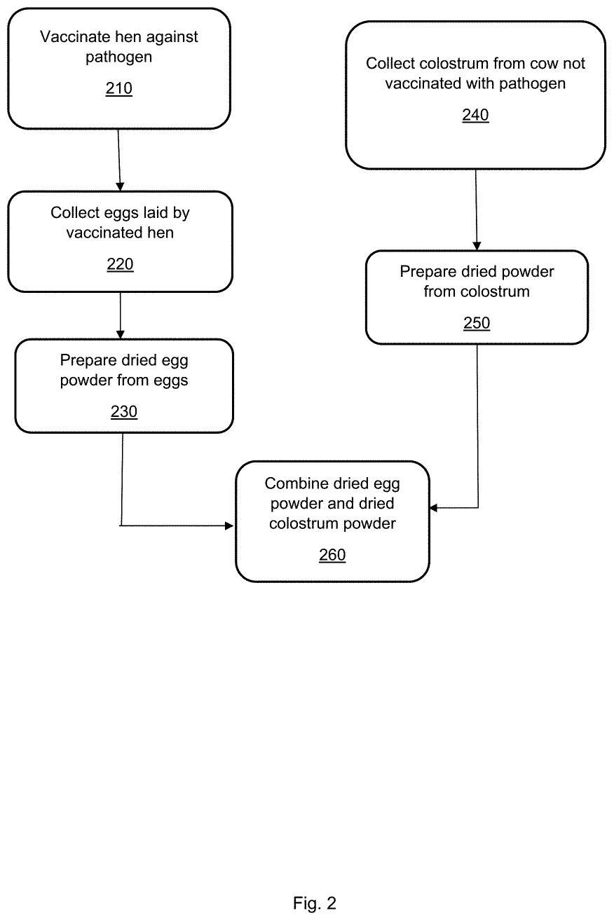

FIGS. 1 and 2 describe embodiments of the preparation of the egg powder and protective protein powder mixture. In FIG. 1, chicken 110 is receiving vaccine 105 which includes multiple antigens associated with pathogens or toxins. Chicken 110 then lays egg 120 which includes antibodies, including antibody 125 inside it. Many antibodies are present in egg 120 although only antibody 125 is depicted for purposes of clarity. Egg 120 is converted to a dry egg powder using techniques described herein. In this embodiment, the protective protein matrix is prepared from non-hyperimmune bovine colostrum. Lactating cow 150 is a non-hyperimmune animal from which colostrum 160 is collected. Colostrum 160 is converted to a dry colostrum powder 170 using techniques described herein. Dry egg powder 130 and dry colostrum powder 170 are combined in ratios disclosed herein to produce therapeutic 180.

FIG. 2 is a flow chart which describes the process depicted in FIG. 1. In step 210, the hens, are vaccinated against one or more pathogens or toxins. After a sufficient time to raise antibodies in response to the vaccine, the hens lay eggs which are collected in step 220. The eggs contain antibodies against the epitopes in the vaccine. In step 230, dried egg powder is prepared from the eggs. In this embodiment, colostrum is used as the protective protein matrix. In step 240, colostrum from a non-hyperimmune cow is collected. Specifically, the cow has not been vaccinated against the targeted pathogens or toxins. The cow is milked to collect colostrum and a dried powder is prepared from the colostrum using techniques described herein (step 250). In step 260, dried egg powder from step 230 is combined with dried colostrum powder from step 250 in ratios disclosed herein to produce the therapeutic composition.

The egg and colostrum (or other matrix protein) mixture may be provided in powdered form. Alternatively, the egg and colostrum mixture may be processed to produce tablets, chewable pills, syrups, elixirs, or drinkable or sprayable aqueous suspensions. Any form known in the art which may be administered to an animal is within the scope of this disclosure. Other additives, including preservatives or flavorings, may be included in the final mixture.

One or more of electrolytes, vitamins, and one or more probiotic cultures may also be included in the therapeutic to further support treatment. Probiotics are microbes that are normally found in the gut. They may be bacteria or yeast. When present in proper amounts, probiotic microbes aid in digestion, inhibit growth of pathogenic organisms, and synthesize nutrients. They may also support the host's immune system or have anti-inflammatory activity. In fact, different probiotic strains provide different benefits to the host. It is for at least this reason that probiotic supplements are often provided as a mixture of multiple strains. The mixture may include a plurality of bacteria strains, a plurality of yeast strains, or a plurality of both bacteria and yeast strains. Such probiotic microbes can benefit from the protection the colostrum or other protein matrix provides to the antibodies in the disclosed therapy. Therefore, a reduced number of colony forming units (CFUs) in each dose of the disclosed therapy may provide the desired efficacy relative to providing probiotic cultures alone. Furthermore, the probiotics may add to the therapeutic effect of the antibodies in the disclosed therapy.

In some embodiments, the microbial strains which may be included as probiotics include one or more of the following list: Enterococcus faecium (including, but not limited to strain SF68), Lactobacillus acidophilus, Lactobacillus casei, Lactobacillus plantarum, Bifidobacterium bifidum, VSL #3, Lactobacillus rhamnosus (including, but not limited to strain GG (LGG)), Bifidobacterium animalis (including, but not limited to, strain AHC7). The latter is reported to be especially effective to combat Clostridium difficile infections of the gut. Other strains known in the art may also be included in the disclosed therapy.

In addition to probiotics, some embodiments may include prebiotics which provide nutrients for the probiotic microbes. The protein matrix may act as a prebiotic. Other prebiotics which may be included are fructooligosaccharides (FOS), beet pulp, raw garlic, dandelion greens, wheat dextrin, chicory, fermented vegetables, and other prebiotics known in the art.

The therapeutic may be provided to a cat, dog, or other animal in doses that may depend on the animal's body weight, the severity of the disease, and whether the therapeutic is being used prophylactically or to treat existing illness. Alternatively, an object can be coated or otherwise treated with a sufficient amount to reduce biofilm formation or allow for targeted application of the coating to a biofilm, membrane, mucosal membrane, or tissue. In an example, a single dose may comprise 3-10 grams of the powdered egg and protein matrix mixture, excluding other additives which may be present in the final product. In some embodiments, a single dose may comprise approximately 5 grams of the powdered egg and protein matrix mixture, excluding other additives which may be present in the final product. The therapeutic may be administered by sprinkling the dry product onto food which the animal may then ingest to contact mucosal membranes or gut tissue. The therapeutic in dry form may be mixed with water or other ingestible liquid and mixed into or decanted onto food which the animal may then ingest. The therapeutic in dry form may be mixed with water or other ingestible liquid and sprayed or otherwise administered into the animal's mouth using a syringe. In some embodiments the animal is allowed to drink the therapeutic composition, or alternatively have it administered directly into the animal's stomach through a nasogastric tube. In some embodiments, the nasogastric tube can be externally coated with the therapeutic agent. In summary, any method of administering the product into an animal or onto an object for insertion into an animal to be treated is within the scope of this disclosure.

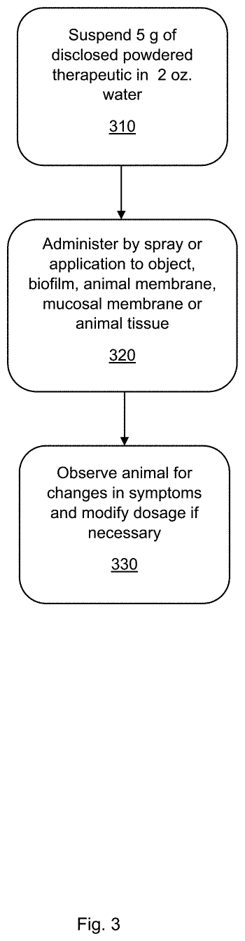

FIG. 3 summarizes an embodiment in which an animal or object is treated with the disclosed composition. In step 310, a user suspends 5 g of the powdered therapeutic as described herein in 2 ounces of water. Additional therapeutic agents, solubilizers, stabilizers, dyes, anti-microbial agents, electrolytes, vitamins, and one or more probiotic or prebiotic cultures or the other useful materials can also be suspended in the composition. The suspended therapeutic is administered, for example, by spray or direct application to an object, biofilm, animal membrane, mucosal membrane, or animal tissue (step 320). The animal can be observed for changes in symptoms, with additional or subsequent applications of the disclosed therapeutic composition in the same, greater, or lesser dosage (step 330).

It is to be understood that the embodiments herein described are merely illustrative. Reference herein to details of the illustrated embodiments is not intended to limit the scope of the claims. The following example is intended to illustrate but not limit the claims.

### Example 1

An example of an antibody preparation according to the instant disclosure was prepared as follows. Chickens (Rhode Island Red) were housed, fed, and cared for according to standard protocol for commercial egg-laying hens. They were raised from hatchlings and fed a high protein diet comprising 20% or more protein for the first 10-15 weeks after hatching. Feed included 2.5-5.0 g calcium per day. Afterwards, the hens were fed a diet comprising less than 20% protein. Overall, the range of protein in the chicken feed was between 14-22 g per day. Adequate water was also provided.

The hens were vaccinated by subcutaneous injection with commercially available animal vaccines against the following diarrhea-causing organisms: coronavirus, C. perfringens, rotavirus, Salmonella, E. coli, Campylobacter, and parvovirus.

Following vaccinations, eggs were collected from the hens. A dehydrated egg powder was prepared from the whole shell eggs by drying the eggs as described herein and grinding the dehydrated product to produce an egg powder.

A sample of the egg powder was sent to an ISO/IEC 17025:2005 accredited commercial testing facility to assess the microbial and heavy metal contents. The laboratory report is provided as Table 1. These results indicate that the egg powder is safe for ingestion.

TABLE 1
Analysis of Egg Powder

| Analysis | Result | Per Unit | Specifications | Method |
|---|---|---|---|---|
| Mercury | <0.001 | ppm | Report | ICP-MS USP<730> |
| Lead | 0.117 | ppm | Report | ICP-MS USP<730> |
| Arsenic | 0.013 | ppm | Report | ICP-MS USP<730> |
| Cadmium | 0.006 | ppm | Report | ICP-MS USP<730> |
| Identification by FTIR | 97.48% positive | | Report | FTIR |
| Total Aerobic Microbial Count | <10 | CFU/g | Report | USP<2021> |
| E. coli | Absent | per 10 g | Absent | USP<2022> |
| Salmonella | Absent | per 10 g | Absent | USP<2022> |
| Coliforms | <3.0 | MPN/g | Report | AOAC 966.24 |
| Staphylococcus aureus | Absent | per 10 g | Absent | USP<2021> |
| Total Yeast and Mold | <10 | CFU/g | <100 CFU/g | USP<2021> |
| Yeast | <10 | CFU/g | <100 CFU/g | |
| Mold | <10 | CFU/g | <100 CFU/g | |

USP = microbial limits preparatory testing;
AOAC = Association of Official Agricultural Chemists
CFU = colony forming units;
ICP-MS = Inductively Coupled Plasma Mass Spectrometry;
FTIR = Fourier transform infrared spectroscopy

### Example 2

Tables 2 and 3 illustrate some mucosal membranes (mucosal surface) which the organisms listed above may infect, the type of pathology they may cause (infection), and the animal in which they are often found. Consequently, Tables 2 and 3 describes animals which may be treated with the disclosed therapeutic/prophylactic, the disease for which the animal may be treated (infection), the site of administration (mucosal surface), and the organisms to which the antibodies may adhere. More specifically, Table 2 describes treatment of dogs, cats, and swine and Table 3 describes treatment of cow and horse.

| Mucosal | | | |
|---|---|---|---|
| Surface | Infection | Dog/Cat | Swine |
| Bronchial | Bronchitis | 1. Streptococcus | 1. Haemophilus parasuis |
| | | zooepidem | 2 . . . Bordetella bronchiseptica |
| | | 2. Bordetella | 3. Pasteurella |
| | | bronchiseptica | |
| | | 3. Pasteurella | |
| Enometrium | Enometritus | 1. Escherichia coli | 1. Staphylococcus hyicus |
| | | 2. streptococci | 2. E coli |
| | | 3. staphylococci | |
| | | 4. Proteus sp | |
| Esophageal | Candidiasis | 1. Malassezia | |
| | Pharyngitis | pachydermatis | |
| Intestinal | Gastroenteritis | 1. Yersinia enterocolitica | 1. Yersinia enterocolitica |
| | Giardia | 4. Giardia duodenalis | 2. Salmonella Typhimurium |
| Nasal | Rhinitis | 1. Aspergillus fumigatus | 1. Bordetella bronchiseptica |
| | Sinusitis | 2. Bordetella | 2. Pasteurella multocida |
| | | bronchiseptica | |
| Vaginal | Vaginitis | 1. Escherichia coli | 1. β-haemolytic streptococci |
| | Yeast | 2. Staphylococcus | 2. Trueperella pyogenes |
| | | 3. Streptococcus | 3. Actinobacillus suis |
| | | 4. Klebsiella | 4. Bacteroides spp. |
| | | 5. Pasteurella | 5. Clostridium spp. |
| | | 6. Pseudomonas | |
| | | 7. Proteus species | |
| Ear | Otitus | 1. Malassezia | 1. Pasteurella multocidia |
| | | pachydermatitis | (PMt) |
| | | 2. Candida | 2. Streptococcus suis |
| | | 3. Malassezia | |
| | | pachydermatis | |
| Urinary | Cystitis | 1. E. coli | 1. Actinobaculum suis |
| | | 2. Klebsiella spp | |
| | | 3. Staphylococcus spp | |
| | | 4. Enterococcus spp | |
| | | 5. Proteus spp. | |
| | | 6. Pseudomonas spp | |
| Anal canal | Proctitis | 1. Scaris lumbricoides | |
| | Anusitis | | |

TABLE 3

| Mucosal | | | |
|---|---|---|---|
| Surface | Infection | Cow | Horse |
| Bronchial | Bronchitis | 1. Bovine Respiratory | 1. Bordetella bronchiseptica |
| | | Syncytial Virus (BRSV) | 2. Pasteurella |
| | | 2. Parainfluenza 3 (PI3) | 3. Pseudomonas aeruginosa |
| | | 3. Adenovirus | |
| | | 4. Bovine Viral Diarrhea | |
| | | Virus (BVDV) | |
| | | 5. Infectious Bovine | |
| | | Rhinotracheitis (IBR)) | |
| | | 6. Pasteurella multocida | |
| | | 7. Mannheimia | |
| | | haemolytica | |
| | | 8. Histophilus somni | |
| | | 9. Mycoplasma bovis | |
| | | 10. Parasitic (lungworm) | |
| | | 11. fungal (Aspergillus) | |
| Enometrium | Enometritus | 1. Trueperella pyogenes | 1. Streptococcus |
| | | 2. Fusobacterium | zooepidemicus |
| | | necrophorum | 2. Escherichia coli |
| | | | 3. Pseudomonas aeruginosa |
| | | | 4. Klebsiella pneumoniae |
| | | | 5. Streptococcus equi subsp |
| | | | zooepidemicus |
| Esophageal | Candidiasis | 1. Candida albicans | 1. Streptococcus equi |
| | Pharyngitis | | 2. zooepidemicus |
| Intestinal | Gastroenteritis | 1. Yersinia enterocolitica | 1. Yersinia enterocolitica |
| | Giardia | 2. Salmonella | 2. Salmonella Typhimurium |
| | | Typhimurium | 3. Escherichia coli |
| Nasal | Rhinitis | 1. Pasteurella multocida | 1. Streptococcus equi |
| | Sinusitis | 2. Escherichia coli | zooepidemicus |
| | | | 2. Actinobacillus equuli |
| | | | 3. Bordetella bronchiseptica |
| | | | 4. Escherichia coli |
| | | | 5. Pasteurella spp |
| | | | 6. Pseudomonas aeruginosa |
| Vaginal | Vaginitis | 1. Ureaplasma | 1. Taylorella equigenitalis |
| | Yeast | ureolyticum | |
| | | 2. Mycoplasma spp | |
| Ear | Otitus | 1. Mannheimia | 1. Streptococcus spp |
| | | haemolytica | |
| | | 2. Pasteurella multocida | |
| | | 3. Histophilus somni | |
| | | 4. Mycoplasma bovis | |
| Urinary | Cystitis | 1. Corynebacterium | 1. Pseudomonas aeruginosaz |
| | | renale | 2 Arcanobacterium spp |
| | | 2. Corynebacterium | 3. Trueperella spp |
| | | cystitidis | |
| | | 3. Escherichia coli | |
| | | 4. Corynebacterium | |
| | | pilosum | |
| Anal canal | Proctitis | | 1. Salmonella |
| | Anusitis | | 2. Clostridium difficile |
| | | | 3. Clostridium perfringens |
| | | | 4. Lawsonia intracellularis |
| | | | 5. Neorickettsia risticii |

1. A composition for treatment of mucosal infection in an animal by application to a mucous membrane, the composition comprising:
a mixture of IgY antibodies specific for a plurality of antigens obtained from a plurality of organisms, wherein the plurality of organisms can cause infection of the mucous membrane; and
a protective matrix comprising non-hyperimmune colostrum combined with the mixture of IgY antibodies, wherein the protective matrix comprises at least 20% by weight of the composition.

2. The composition of claim 1, wherein the mixture of IgY antibodies is obtained from one or more eggs laid by one or more birds, wherein each of the one or more birds have been vaccinated with the plurality of antigens.

3. The composition of claim 1, wherein the non-hyperimmune colostrum is obtained from a bovine.

4. The composition of claim 1, wherein the plurality of organisms can form a biofilm.

5. The composition of claim 1, wherein the composition can be administered as a solid, a powder, a powder suspended liquid, a cream, as a liquid, by mist, aerosol, or spray, or coated on an object insertable into an animal.

6. The composition of claim 1, wherein the plurality of organisms can cause at least one of the following mucous membrane infections: oral thrush, canker sores, bronchitis, endometritis, candidiasis, pharyngitis, gastroenteritis, giardia infection, chlamydia, gonorrhea, rhinitis, sinusitis, vaginitis, vaginal yeast infection, otitis, otitis media, group B streptococcus infection of the vagina or rectum, oral or genital herpes, vaginal yeast infection, cystitis, urinary tract infection, proctitis, paracoccidioidomycosis, histoplasmosis, mucormycosis, candida, group A streptococcal infection of the pharynx, respiratory syncytial virus (RSV) infection, and anusitis.

7. The composition of claim 1, wherein the plurality of organisms are at least one of the following: Streptococcus zooepidem, Bordetella bronchiseptica, Pasteurella, Haemophilus parasuis, Bovine Respiratory Syncytial Virus (BRSV), Parainfluenza 3 (PI3), Adenovirus, Bovine Viral Diarrhea Virus (BVDV), Infectious Bovine Rhinotracheitis (IBR)), Pasteurella multocida, Mannheimia haemolytica, Histophilus somni, Mycoplasma bovis, Parasitic organisms (e.g. lungworm), fungal organisms (e.g. Aspergillus), Pseudomonas aeruginosa, Staphylococcus hyicus, Escherichia coli, Trueperella pyogenes, Fusobacterium necrophorum, Streptococcus zooepidemicus, Klebsiella pneumoniae, Streptococcus equi subsp zooepidemicus, Candida albicans, Streptococcus equi, zooepidemicus, Yersinia enterocolitica, Salmonella Typhimurium, Pasteurella multocida, Streptococcus equi zooepidemicus, Actinobacillus equuli, Pasteurella spp, β-haemolytic streptococci, Trueperella pyogenes, Actinobacillus suis, Bacteroides spp., Clostridium spp., Ureaplasma ureolyticum, Mycoplasma spp, Taylorella equigenitalis, Pasteurella multocidia (PMt), Streptococcus suis, Mannheimia haemolytica, Pasteurella multocida, Histophilus somni, Mycoplasma bovis, Streptococcus spp., Corynebacterium renale, Corynebacterium cystitidis, Corynebacterium pilosum, Pseudomonas aeruginosaz, Arcanobacterium spp., Trueperella spp., Salmonella, Clostridium difficile, Clostridium perfringens, Lawsonia intracellularis, and Neorickettsia risticii.

8. A method of treating or preventing infection, comprising:
providing an antibody mixture comprising a non-hyperimmune colostrum and antibodies produced by an avian animal to act against an organism that infects a mucous membrane; and
applying the antibody mixture to the mucous membrane.

9. The method of treating or preventing infection of claim 8, wherein the avian animal antibodies comprise a mixture of IgY antibodies obtained from one or more eggs laid by one or more birds, wherein each of the one or more birds have been vaccinated with the plurality of antigens.

10. The method of treating or preventing infection of claim 8, wherein the non-hyperimmune colostrum is obtained from a bovine.

11. The method of treating or preventing infection of claim 8, wherein the plurality of organisms can form a biofilm.

12. The method of treating or preventing infection of claim 8, wherein the composition can be administered as a solid, a powder, a powder suspended liquid, a cream, as a liquid, by mist, aerosol, or spray, or coated on an object insertable into an animal.

13. The method of treating or preventing infection of claim 8, wherein the plurality of organisms can cause at least one of the following mucous membrane infections: oral thrush, canker sores, bronchitis, endometritis, candidiasis, pharyngitis, gastroenteritis, giardia infection, chlamydia, gonorrhea, rhinitis, sinusitis, vaginitis, vaginal yeast infection, otitis, otitis media, group B streptococcus infection of the vagina or rectum, oral or genital herpes, vaginal yeast infection, cystitis, urinary tract infection, proctitis, paracoccidioidomycosis, histoplasmosis, mucormycosis, candida, group A streptococcal infection of the pharynx, respiratory syncytial virus (RSV) infection, and anusitis.

14. The method of treating or preventing infection of claim 8, wherein the plurality of organisms are at least one of the following: Streptococcus zooepidem, Bordetella bronchiseptica, Pasteurella, Haemophilus parasuis, Bovine Respiratory Syncytial Virus (BRSV), Parainfluenza 3 (PI3), Adenovirus, Bovine Viral Diarrhea Virus (BVDV), Infectious Bovine Rhinotracheitis (IBR)), Pasteurella multocida, Mannheimia haemolytica, Histophilus somni, Mycoplasma bovis, Parasitic organisms (e.g. lungworm), fungal organisms (e.g. Aspergillus), Pseudomonas aeruginosa, Staphylococcus hyicus, Escherichia coli, Trueperella pyogenes, Fusobacterium necrophorum, Streptococcus zooepidemicus, Klebsiella pneumoniae, Streptococcus equi subsp zooepidemicus, Candida albicans, Streptococcus equi, zooepidemicus, Yersinia enterocolitica, Salmonella Typhimurium, Pasteurella multocida, Streptococcus equi zooepidemicus, Actinobacillus equuli, Pasteurella spp, β-haemolytic streptococci, Trueperella pyogenes, Actinobacillus suis, Bacteroides spp., Clostridium spp., Ureaplasma ureolyticum, Mycoplasma spp, Taylorella equigenitalis, Pasteurella multocidia (PMt), Streptococcus suis, Mannheimia haemolytica, Pasteurella multocida, Histophilus somni, Mycoplasma bovis, Streptococcus spp., Corynebacterium renale, Corynebacterium cystitidis, Corynebacterium pilosum, Pseudomonas aeruginosaz, Arcanobacterium spp., Trueperella spp., Salmonella, Clostridium difficile, Clostridium perfringens, Lawsonia intracellularis, and Neorickettsia risticii.

15. A composition for therapeutic treatment of biofilms, the composition comprising:
a mixture of IgY antibodies specific for a plurality of antigens obtained from a plurality of biofilm forming organisms, wherein the plurality of biofilm forming organisms can cause infection; and
a protective matrix comprising non-hyperimmune colostrum combined with the mixture of IgY antibodies, wherein the protective matrix comprises at least 20% by weight of the composition.

16. The composition of claim 15, wherein the mixture of IgY antibodies is obtained from one or more eggs laid by one or more birds, wherein each of the one or more birds have been vaccinated with the plurality of antigens.

17. The composition of claim 15, wherein the non-hyperimmune colostrum is obtained from a bovine.

18. A method of treating or preventing biofilm formation, comprising:
providing an antibody mixture comprising a non-hyperimmune colostrum and antibodies produced by avian animal to act against a biofilm forming organism; and
applying the antibody mixture to the biofilm or a biofilm support.

19. The method of treating or preventing biofilm formation of claim 18, wherein the biofilm support is a mucous membrane.

20. The method of treating or preventing biofilm formation of claim 18, wherein the biofilm support is insertable into an animal.
